(19) United States
(12) Patent Application Publication
(10) Pub. No.: US 2014/0060541 A1
(43) Pub. Date: Mar. 6, 2014

(54) METHOD AND SYSTEM FOR EVALUATION OF PATIENT BREATHING STAGES

(71) Applicants: Robert T. Boyer, Longmont, CO (US); Gary S. Milne, Louisville, CO (US)

(72) Inventors: Robert T. Boyer, Longmont, CO (US); Gary S. Milne, Louisville, CO (US)

(73) Assignee: NELLCOR PURITAN BENNETT LLC, Boulder, CO (US)

(21) Appl. No.: 13/604,905

(22) Filed: Sep. 6, 2012

(51) Int. Cl. A61M 16/00

(52) U.S. Cl. 128/204.23

(57) ABSTRACT

The disclosure relates generally to a method and a system for acquiring patient parameters and indicating the status of patient parameter relationship expressions for a selected breathing stage from a plurality of breathing stages.

### TECHNICAL FIELD

The disclosure relates generally to a method and a system for monitoring a patient's unassisted breathing readiness. More particularly, the disclosure relates to a method and a system for indicating breathing stages of a patient.

### BACKGROUND

Ventilators provide pressurized respiratory gases to patients to assist their breathing. Respiratory gases may include fresh air, scrubbed air, and anesthetics, for example. As the patient conditions change, the patient gradually transitions from assisted to unassisted breathing. A patient's readiness for withdrawal from mechanical ventilation is typically indicated by the successful completion of a spontaneous breathing trial (SBT). The discontinuation of assisted ventilation process may require as much as 42% of the time that a patient spends on a mechanical ventilator. Explicitly performed SBTs, which are performed by a clinician, limit the frequency at which a patient may be tested. Unnecessary delays in the discontinuation process may increase the complication rate from mechanical ventilation, e.g. pneumonia and airway trauma.

Even further, it may be beneficial to determine when a patient is not ready to breathe unassisted. Aggressiveness in the discontinuation process can cause additional problems, such as difficulty in reestablishing artificial airways and compromising gas exchange. More favorable patient outcomes and lower costs may be possible by earlier transition to unassisted breathing.

### SUMMARY

A method and a system to evaluate breathing stages representing levels of readiness of a patient to breathe unassisted are provided herein. In one embodiment of the system according to the disclosure, the system includes a computer readable storage medium having stored therein a plurality of patient parameter relationship expressions, each patient parameter relationship expression based on a patient parameter and an operator. The system also includes a computing device, coupled to the computer readable storage medium, that is configured to acquire at least some of the patient parameters, and further includes a user interface, coupled to the computer readable storage medium, that is configured to (a) select one or more patient parameter relationship expression from the plurality of patient parameter relationship expressions for each of a plurality of breathing stages; (b) select a breathing stage from the plurality of breathing stages; and (c) present a view on a display device, the view including status indicators operable to indicate a status of each of the one or more patient parameter relationship expression for the selected breathing stage based on the acquired patient parameters.

The above-mentioned and other disclosed features which characterize the embodiments of the system and method described herein advantageously enable standardization of the process of, and protocols for, evaluating patient parameters and determining breathing transition stages, i.e. from readiness to be weaned to readiness to be extubated. Another advantage is that the system and method will enable clinicians to determine that a patient is ready to transition to the next stage(s) without waiting for daily rounds, thereby reducing the amount of time a patient is ventilated. Even further, as standardized data is acquired over time, the transition protocols may be improved, reducing even further the amount of time patients are ventilated and/or reducing the complications that arise from premature or delayed discontinuation of ventilation. In addition to the patient care benefits, a further benefit is the reduction of costs associated with ventilating a patient when assisted ventilation is not required.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some or none of the enumerated advantages.

### BRIEF DESCRIPTION OF THE DRAWINGS

The above-mentioned and other disclosed features, and the manner of attaining them, will become more apparent and will be better understood by reference to the following description of disclosed embodiments taken in conjunction with the accompanying drawings, wherein:

FIG. 1 is a block diagram of an embodiment of a system according to the disclosure for evaluation of patient breathing stages;

FIG. 2 is a flowchart of an embodiment of a method according to the disclosure for evaluation of patient breathing stages;

FIG. 3 is a block diagram of an embodiment of a software product according to the disclosure operable for evaluation of patient breathing stages;

FIG. 4 is a block diagram of another embodiment of a system according to the disclosure for evaluation of patient breathing stages; and

FIGS. 5-9 are exemplary views of an embodiment of a user interface according to the disclosure; and

Corresponding reference characters indicate corresponding parts throughout the several views. Although the drawings represent embodiments of various features and components according to the present disclosure, the drawings are not necessarily to scale and certain features may be exaggerated in order to better illustrate and explain the present invention. The exemplification set out herein illustrates embodiments of the disclosure, and such exemplifications are not to be construed as limiting the scope of the invention in any manner.

### DETAILED DESCRIPTION

For the purposes of promoting an understanding of the principles of the invention, reference will now be made to the embodiments illustrated in the drawings, which are described below. The embodiments disclosed below are not intended to be exhaustive or limit the invention to the precise form disclosed in the following detailed description. Rather, the embodiments are chosen and described so that others skilled in the art may utilize their teachings. It will be understood that no limitation of the scope of the disclosure is thereby intended. The invention includes any alterations and further modifications in the illustrated devices and described methods and further applications of the principles of the invention which would normally occur to one skilled in the art to which the invention relates.

The transitional term “comprising”, which is synonymous with “including,” or “containing,” is inclusive or open-ended and does not exclude additional, unspecified elements or method steps. By contrast, the transitional term “consisting” is a closed term which does not permit addition of unspecified terms.

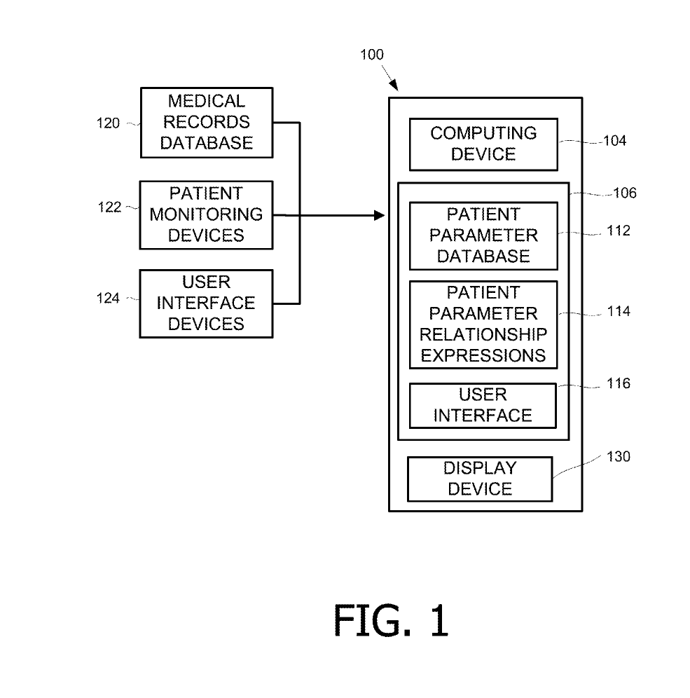

Referring to FIG. 1, a block diagram of an embodiment of a breathing stage evaluation system according to the disclosure is presented. In the present embodiment, the system is denoted by numeral 100. System 100 may receive patient parameters from a plurality of data sources including a medical records database, patient monitoring devices, and user interfaces. The medical records database may contain patient information such as age and medical condition, and patient parameters based on, for example, laboratory analysis of the patient's fluids. Patient monitoring devices automatically record patient parameters such as heart rate and blood pressure, for example. Patient monitoring devices may be included in, or be part of, a mechanical ventilator. User interfaces enable entry of patient parameters based on, for example, observation of the patient's state or performance of patient maneuvers.

System 100 comprises a computing device 104 and a computer readable storage medium 106 having stored therein a patient parameter database 112, patient parameter relationship expressions 114 and a user interface 116. A medical records database 120, patient monitoring devices 122 and user interface devices 124, are shown coupled to system 100. Computing device 104 may comprise one or more processing device and software programs configured to cause the processing device to acquire patient parameters from the data sources and to store the patient parameters in patient parameter database 112. Each patient parameter relationship expression describes a relationship between at least one patient parameter and a threshold. The relationship comprises an operator and may comprise a single parameter (e.g. A=c, where A is the parameter and “c” is a threshold), more than one parameter (e.g. A+B=c, where A and B are parameters and “c” is a threshold), a function (e.g. f(A)=c, where f(A) is any function of parameter A and “c” a threshold), and any combination of the foregoing. Of course, the operator may represent an inequality as well as an equality relationship. The system may comprise functions which users may include in the expressions. For example, a function may be provided to convert a parameter, and the conversion function may then be used in a patient parameter relationship expression. The expressions may also include ranges, e.g. {A<K+2B≦c}, where A and B are parameters, K is a constant and “c” is a threshold; therefore, the expression may be satisfied by a range of values for A in which A is less than K. Ranges may also be defined in a function comprising Boolean algebra, where the threshold is a logical outcome, e.g. yes/no. Expressions may also include functions representing time durations or compliance requirements. For example, an expression may include a function requiring that a parameter exceed a threshold for a predetermined time during the trial or falls within a range for a predetermined time.

The status of a patient parameter relationship expression is determined by comparing the result of the function to the threshold based on the operator. Thus, the patient parameter relationship expression may be satisfied if the condition is satisfied or unsatisfied if the condition is not satisfied. In the event that the patient parameter is not yet available, the patient parameter relationship expression status may indicate that the evaluation of the expression is incomplete. In the present context, the terms satisfied, unsatisfied and incomplete are only exemplary. Any other suitable terms may be used to denote the status of an expression, protocol or trial. For example, Boolean logic terms such as true/false or yes/no may be used instead of satisfied and unsatisfied.

System 100 may also comprise one or more software programs configured to cause computing device 104 to determine the status of patient parameter relationship expressions 114 based on the patient parameters and to present corresponding status indicators with user interface 116. User interface 116 may comprise a text or graphical user interface operable to present the status indicators in a predetermined manner with a display device 130. User interface 116 may also be operable to define expressions and protocols, select protocols for evaluating breathing stages, modify the protocols, and associate the protocols with patients. A protocol may be associated with a patient by selecting the patient, selecting the protocol, and then saving or storing the patient configuration file. Exemplary status indicators may comprise icons of different colors or shapes, flashing icons, text messages, audible indications, and any other means for providing constructive notice to the clinician concerning the success or failure of the trial.

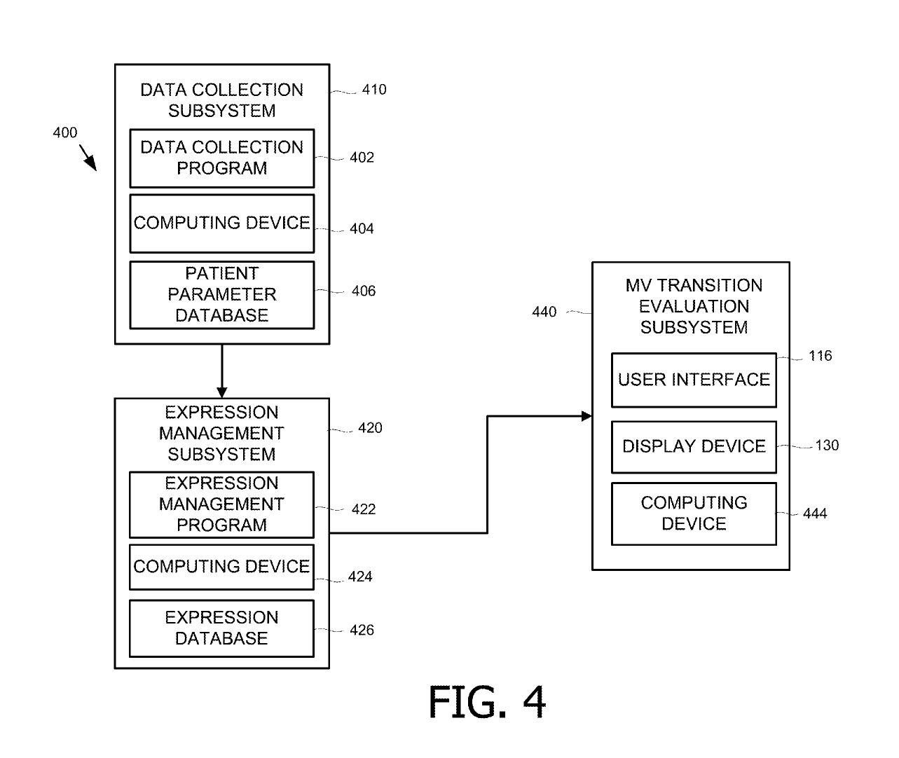

The components of system 100 may be integrated or distributed. A distributed system is described below with reference to FIG. 4.

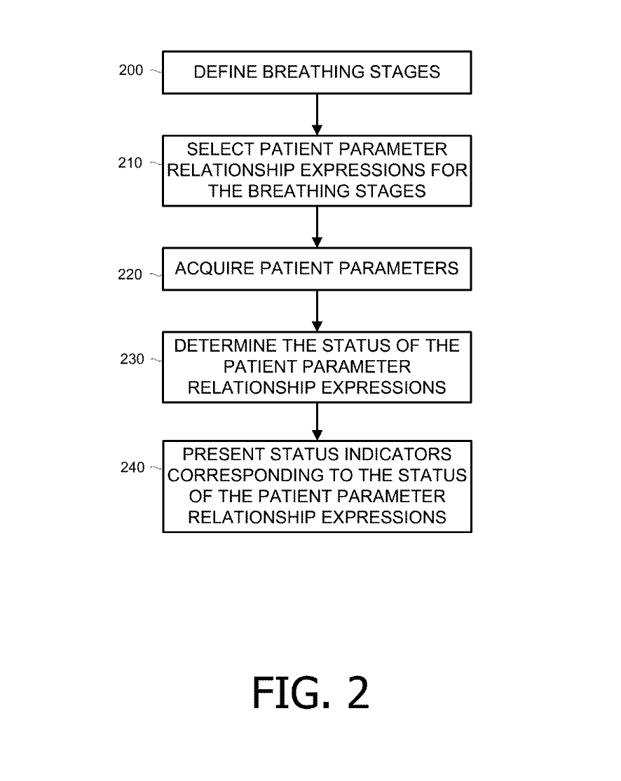

An embodiment according to the disclosure of a method for evaluating breathing stages of a patient will now be described with reference to FIG. 2. The method may be performed, at least partially, with system 100. At 200, breathing stages representing levels of readiness of the patient to breathe unassisted are defined. Exemplary breathing stages include readiness to be weaned from MV, readiness to perform SBT, tolerance or failure of SBT performance, and readiness to be extubated. Each breathing stage may include success criteria or both success and failure criteria.

In one variation, readiness to perform a SBT may comprise two stages, an automatically detected stage and a patient evaluation stage. In the automatically detected stage, the system evaluates the patient parameter expressions and determines if the stage has been successfully completed. In the patient evaluation stage, the patient is evaluated and successful completion of the stage is based, at least in part, on the results of the evaluation. The evaluation may comprise an interaction with the patient. For example, a clinician may require the patient to perform a maneuver, e.g. a negative inspiratory force (NIF) maneuver, or check the patient's awareness to confirm that the patient is not affected by neuro-muscular blocking agents. In one example, satisfaction of the automatically detected stage is a pre-condition for the patient evaluation stage. In the present example, the clinician may not be prompted to evaluate the patient until certain conditions are satisfied. In another example, the patient evaluation stage is performed first, and when the automatically detected stage is subsequently satisfied, the readiness to perform SBT stage is satisfied. In the present example, the clinician evaluates the patient when convenient or appropriate, and the automatically detected stage serves a confirmation or validation function.

At 210, patient parameter relationship expressions are selected for each of the breathing stages. In one variation, a plurality of protocols are defined, each protocol including one or more patient parameter relationship expressions. In another variation, a protocol may comprise a composite expression comprised of multiple parameters and operators. Patient parameter relationship expressions may be selected by selecting an appropriate protocol. For each breathing stage, potentially multiple protocols may be defined (e.g., a well known protocol found in research literature, or a doctor's or hospital's own variant protocol). Each protocol is represented as a series of patient parameter relationship expressions that may be loaded from a configuration file. The protocols may be user configured (e.g., users may add, modify, or delete expressions and protocols as desired—for example to differentiate adult ICU patients having been briefly under anesthesia from a pediatric case from a long-term mechanically ventilated patient). A protocol may include success criteria or both success and failure criteria. Success criteria for a protocol may comprise satisfaction of success expressions. Failure criteria for a protocol may comprise satisfaction of failure expressions.

In one variation, user interface 116 may be operable to define expressions and protocols, select protocols for evaluating breathing stages, and modify the protocols. In one example, a number of protocols are predefined. A patient is selected, predefined protocols are selected, and the predefined protocols are then associated with the patient. If a predefined protocol is modified after it is associated with the patient, the modifications may be stored in a patient configuration file. Also, the modifications may be stored in a new protocol. The breathing stage evaluation system may support evaluation of multiple patients by enabling a clinician to switch patients, for example by selecting a different patient from a drop-down list. When the different patient is selected, the different patient's configuration file restores the previously selected protocols for the patient.

Once the patient's protocols are defined, at 220, patient parameters are acquired. Patient parameters may be acquired by the breathing stage evaluation system at periodic time intervals, based on the patient's physiology, or upon occurrence of some predetermined event. Patient parameters may be acquired for the present breathing stage of the patient, in accordance with the patient parameter relationship expressions selected for the present breathing stage of the patient. Alternatively, all the patient parameters associated with a patient may be acquired concurrently.

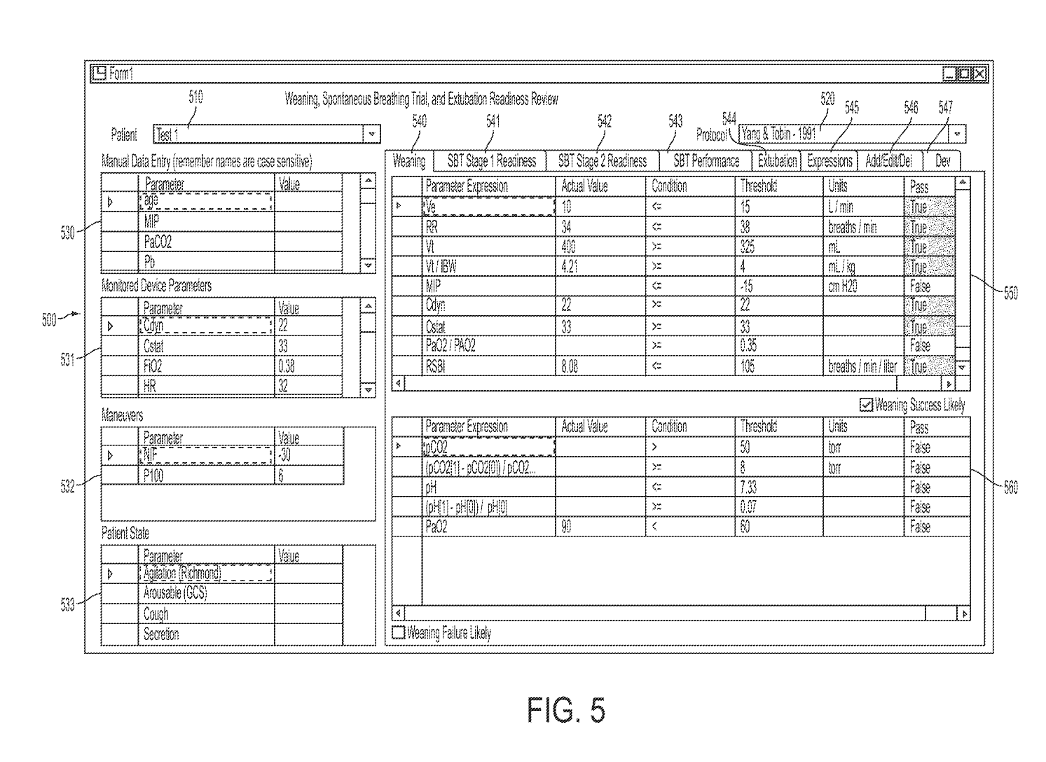

Once the patient parameters for the patient parameter relationship expressions have been acquired, at 230, the status of the patient parameter relationship expressions are determined. As described above, the breathing stage evaluation system may determine the status of a patient parameter relationship expression by comparing the patient parameter (or a function based on the patient parameter) to the threshold based on an operator. An exemplary expression for parameter Ve is shown on FIG. 5, where the operator is and the threshold is 15 L/min. Accordingly, the expression is satisfied if the patient parameter Ve is less than or equal to 15. As shown, Ve=10 and the expression is satisfied (or true).

At 240, status indicators corresponding to the status of the patient parameter relationship expressions are presented with a user interface. Exemplary status indicators include text, graphical images, colors, and combinations thereof.

In one variation, a clinician may check a checkbox to record the likelihood of success or failure of the breathing stage. The clinician may also select another breathing stage and evaluate, based on the status indicators, the status of the stage. In this manner, the clinician may navigate from stage to stage and perform the evaluations required by each stage's protocol, if any. Further, as the patient advances from one stage to the next, as evidenced by the satisfaction of the expressions, the clinician may perform a spontaneous breathing trial or extubate the patient with confidence based on protocols refined over time and tailored for particular patient physiologies. Additionally, the clinician may change protocols, if desired, based on the clinician's evaluation of patient conditions and/or the status of the expressions.

In another variation, the system may automatically indicate the likelihood of success or failure of the breathing stage. In one example, the system may check a checkbox indicative of the likelihood of success or failure of the breathing stage. The system may determine the likelihood of success or failure based on the satisfaction of the success and failure expressions. As before, the clinician may also select another breathing stage and may navigate from stage to stage and perform the evaluations required by each stage's protocol, if any. Further, the clinician may perform a spontaneous breathing trial or extubate the patient.

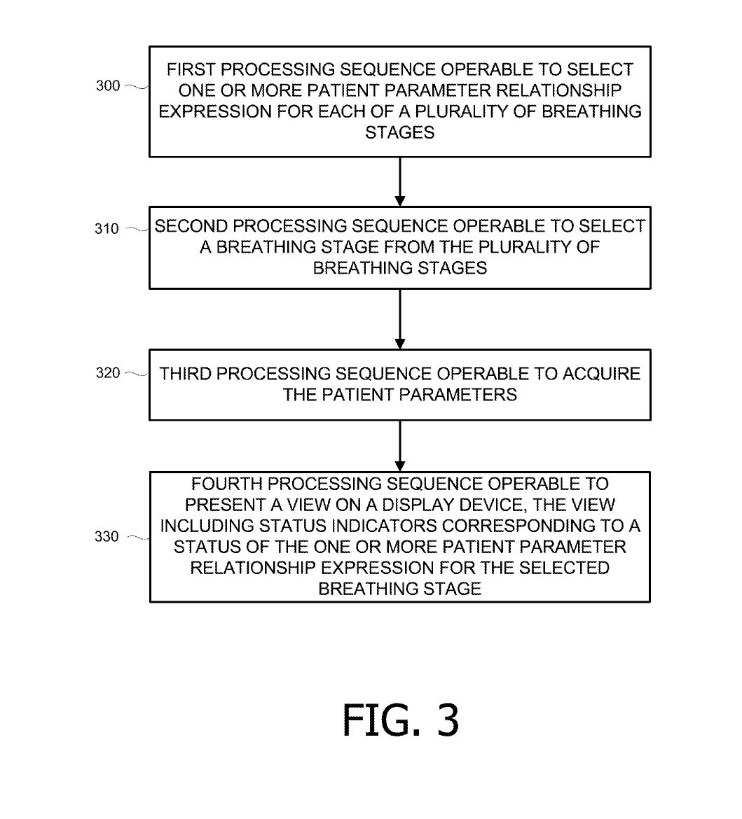

An embodiment according to the disclosure of computer program product for evaluating breathing stages of a patient will now be described with reference to FIG. 3. The computer program product comprises a plurality of computer readable processing sequences embodied in a computer readable storage medium and operable to present views of a user interface with a display device and to receive user inputs associated with the views of the user interface, thereby enabling a user to manipulate information. Further, the computer program product may be operable to acquire patient parameters, evaluate expressions and update status indicators to enable the clinician to visually evaluate the breathing stage of the patient. The breathing stages, expressions and protocols may be predefined. Also, the computer program product may be operable to add, modify and delete breathing stages, expressions and protocols.

A first processing sequence 300 is operable to select one or more patient parameter relationship expression for each of a plurality of breathing stages, each of the one or more patient parameter relationship expression based on a patient parameter. A patient parameter relationship expression may be selected by selecting an expression from a list of expressions or by selecting a protocol, each protocol including previously selected expressions, from a list of protocols. Any number patient parameter relationship expressions and protocols may be predefined based on experience or medical literature.

First processing sequence 300 may store the user selections in a configuration file such as a text file, an XML file or any other suitably formatted data structure. The user selections may be stored in a file corresponding to a specific patient. The user selections may also be stored in a database in which the selections are related or associated with the patient. In one variation, first processing sequence 300 presents a selection tool to enable the user to select a configuration file from memory. Once the user selects a configuration file, first processing sequence 300 may receive further user selections corresponding to the breathing stage and the patient parameter relationship expressions of a patient. First processing sequence 300 then stores the modified configuration file as a patient configuration file.

A second processing sequence 310 is operable to select a breathing stage from the plurality of breathing stages.

A third processing sequence 320 is operable to acquire the patient parameters. As described above, patient parameters may be received at periodic time intervals, based on the patient's physiology, or upon occurrence of some predetermined event.

A fourth processing sequence 330 is operable to present a view on a display device, the view including status indicators corresponding to a status of the one or more patient parameter relationship expression for the selected breathing stage. Fourth processing sequence 330 may be operable to compute the status of the one or more patient parameter relationship expression for the selected breathing stage. Exemplary status indicators include text, graphical images, colors, and combinations thereof. The clinician may navigate between stages and check checkboxes to evidence satisfaction of a stage. In one example, satisfaction of a stage automatically selects the succeeding stage for presentation with the display device.

Referring again to FIG. 1, system 100 comprises a computing device 104 including one or more processing device and software programs. As used herein, a software program, algorithm, or processing sequence, is a self consistent sequence of instructions that can be followed to perform a particular task. Software programs may use data structures for both inputting information and performing the particular task. Data structures greatly facilitate data management. Data structures are not the information content of a memory, rather they represent specific electronic structural elements which impart a physical organization on the information stored in memory. More than mere abstraction, the data structures are specific electrical or magnetic structural elements in memory which simultaneously represent complex data accurately and provide increased efficiency in computer operation.

A processing or computing system or device may be a specifically constructed apparatus or may comprise general purpose computers selectively activated or reconfigured by software programs stored therein. The computing device, whether specifically constructed or general purpose, has at least one processing device, for executing machine instructions, which may be grouped in processing sequences, and access to computer readable storage media, or memory, for storing instructions and other information. Many combinations of processing circuitry and information storing equipment are known by those of ordinary skill in these arts. A processing device may be a microprocessor, a digital signal processor (DSP), a central processing unit (CPU), or other circuit or equivalent capable of interpreting instructions or performing logical actions on information. Memory includes both volatile and non-volatile memory, including temporary and cache, in electronic, magnetic, optical, printed, or other format used to store information. Exemplary processing systems include workstations, personal computers, portable computers, portable wireless devices, mobile devices, and any device including a processor, memory and software. Processing systems encompass one or more computing devices and include computer networks and distributed computing devices.

As used herein, a computer network, or network, is a system of computing systems or computing devices interconnected in such a manner that messages may be transmitted between them. Typically one or more computers operate as a “server”, a computer with access to large storage devices such as hard disk drives and communication hardware to operate peripheral devices such as printers, routers, or modems. Other computers, termed “clients”, provide a user interface so that users of computer networks can access the network resources, such as shared data files, common peripheral devices, and inter workstation communication. User interfaces comprise software working together with user devices to communicate user commands to the processing system. Exemplary user devices include touch-screens, keypads, mice, voice-recognition logic, imaging systems configured to recognize gestures, and any known or future developed hardware suitable to receive user commands.

Embodiments of the disclosure may be implemented in “object oriented” software, and particularly with an “object oriented” operating system. The “object oriented” software is organized into “objects”, each comprising a block of computer instructions describing various procedures to be performed in response to “messages” sent to the object or “events” which occur with the object. Such operations include, for example, the manipulation of variables, the activation of an object by an external event, and the transmission of one or more messages to other objects. Messages are sent and received between objects having certain functions and knowledge to carry out processes. Messages are generated in response to user instructions, for example, by a user activating an icon with a mouse pointer or touch-screen to generate an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access.

Referring now to FIG. 4, a block diagram of another embodiment of a breathing stage evaluation system according to the disclosure is presented. In the present embodiment, the system is denoted by numeral 400. As in system 100, system 400 may receive patient parameters from a plurality of data sources including a medical records database, patient monitoring devices, and user interfaces. As shown, system 400 comprises a data collection subsystem 410. Data collection subsystem 410 includes a data collection program 402, a computing device 404 and a patient parameter database 406. Data collection program 402 causes computing device 404 to acquire patient parameters from the data sources and to store the patient parameters in patient parameter database 406. Data collection subsystem 410 may also store monitoring device setting information. Patient parameters may be normalized prior to being stored in patient parameter database 406.

System 400 also comprises an expression management subsystem 420. expression management subsystem 420 includes an expression management program 422, a computing device 424 and an expression database 426. Expression management program 422 may cause computing device 424 to acquire patient parameters from patient parameter database 406 and to store the patient parameters in a computer readable storage medium.

System 400 also comprises an MV transition evaluation subsystem 440. As shown, MV transition evaluation subsystem 440 comprises user interface 116, display device 130 and a computing device 444. MV transition evaluation subsystem 440 may be operable to communicate with expression management program 422 information relating to the patient parameter relationship expressions and patient parameters. MV transition evaluation subsystem 440 may then determine the status of the patient parameter relationship expressions and present a plurality of user interface views with display device 130. The views may be configured to constructively inform a clinician regarding the status of the expressions. User interface 116 may be operable to cause computing device 444 to receive user instructions, such as protocol and expression selections, and to configure and modify expressions and protocols as described above and further below.

In a further embodiment of a breathing stage evaluation system according with the disclosure, data collection subsystem 400, expression management subsystem 420 and MV transition evaluation subsystem 440 are configured in a client/server architecture. In one example, data collection subsystem 400 is integrated in a hospital environment and acquires patient information as determined by the data acquisition parameters of the data sources. Expression management subsystem 420 pulls and stores patient parameters from data collection subsystem 400. Thus, by mapping parameters related to breathing stages, expression management subsystem 420 may be linked to a data collection subsystem 400 which forms part of a hospital's data infrastructure. Furthermore, MV transition evaluation subsystem 440 may be communicatively linked to expression management subsystem 420 to provide a visual aid to a clinician near the patient. Expression management subsystem 420 may function as a server to support a plurality of MV transition evaluation subsystem 440 clients.

In one variation of the present embodiment, expression management program 422 may also determine the status of the expressions and store the status of the expressions in expression database 426. In the present variation, MV transition evaluation subsystem 440 may receive the status information and present status indicators accordingly.

In a further embodiment of a breathing stage evaluation system, data collection subsystem 410 includes expression database 426 and expression management subsystem 420 is not required. In the present embodiment, MV transition evaluation subsystem 440 is communicatively coupled with data collection subsystem 410.

A breathing stage evaluation system according with the disclosure may present a plurality of views corresponding to a plurality of breathing stages. Each view may be presented on a separate tab. A view may include a drop-down list of patients that may be selected to enable a clinician to switch the presentation of information for different patients, a drop-down list of protocols to enable a clinician to quickly switch from one protocol to another within the given breathing stage. A view may include data entry fields to enter patient parameters. A view may also include one or more indicators to provide constructive notice of the success or failure of patient parameter expressions or the particular stage.

Exemplary user interface views are presented on FIGS. 5-8. FIG. 5 presents a user interface view 500 comprising a patient selection box 510, a protocol selection box 520, a plurality of patient parameter panels 530-533, and a plurality of tabs 540-547. Tab 540, corresponding to the weaning stage, is selected. Patient selection box 510 and protocol selection box 520 enable users to select patients and protocols. The selections may be stored in patient specific configuration files on expression database 426 or on any other suitable storage location. Patient parameter panels 530-533 display patient parameters corresponding to, respectively, manual data entry, monitored devices, maneuvers, and patient states. A user may enter manually a patient parameter for parameters shown in panels 530, 532 and 533.

Weaning tab 540 includes a weaning success likely table 550 and a weaning failure likely table 560. The success likely table comprises success expressions and the failure likely table comprises failure expressions. Each table also includes a column labeled “pass” showing the status indicators corresponding to the present status of the expressions. The plurality of expressions represent a protocol. Each expression is presented as a line item, detailing the expression, parameter value or calculated result of the function based on the parameter, the condition specified, and the threshold (target) value. As shown, status indicators include text (true/false) and a color indicative of success (evidenced by colored “True” status indicators in table 550). Satisfaction of an expression may be highlighted green, non-satisfaction may be highlighted red, and unable to compute may be left plain. Furthermore, weaning tab 540 displays a weaning success likely checkbox and a weaning failure likely checkbox. In one example, the system automatically shows a checkmark in the appropriate checkbox to indicate the likelihood of weaning success or failure based on the success or failure of the patient parameter expressions. The system may automatically determine that success is likely if all the success expressions are satisfied and none of the failure expressions are satisfied. The system may automatically determine that failure is likely a failure expression is satisfied. In another example, a clinician may check the appropriate checkbox to indicate the likelihood of weaning success or failure based on the success or failure of the patient parameter expressions.

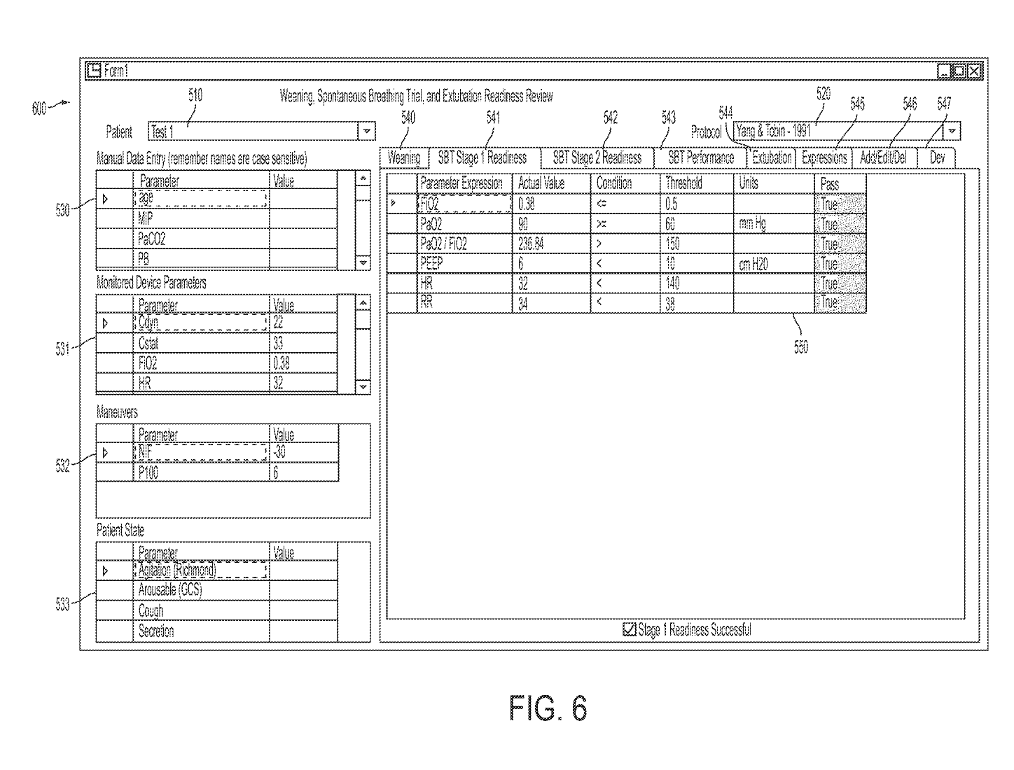

FIG. 6 presents a user interface view 600 with a tab labeled “SBT Stage 1 Readiness” selected. In the present example, satisfaction of the success expressions may represent readiness to conduct a spontaneous breathing test. In another example, in which the stage comprises success and failure expressions, satisfaction of the success expressions and lack of satisfaction of failure expressions may represent readiness to conduct a spontaneous breathing test. FIG. 6 also shows an SBT Stage 2 Readiness tab 542 and an SBT Performance tab 543, which are not selected. In one variation, stage 1 is an automatically detected stage and stage 2 is a patient evaluation stage. Thus, in stage 2 the patient is evaluated (e.g. by the clinician) and successful completion of the stage is based, at least in part, on the results of the evaluation. As discussed previously, stage 1 and stage 2 should be satisfied before proceeding to the SBT Performance stage.

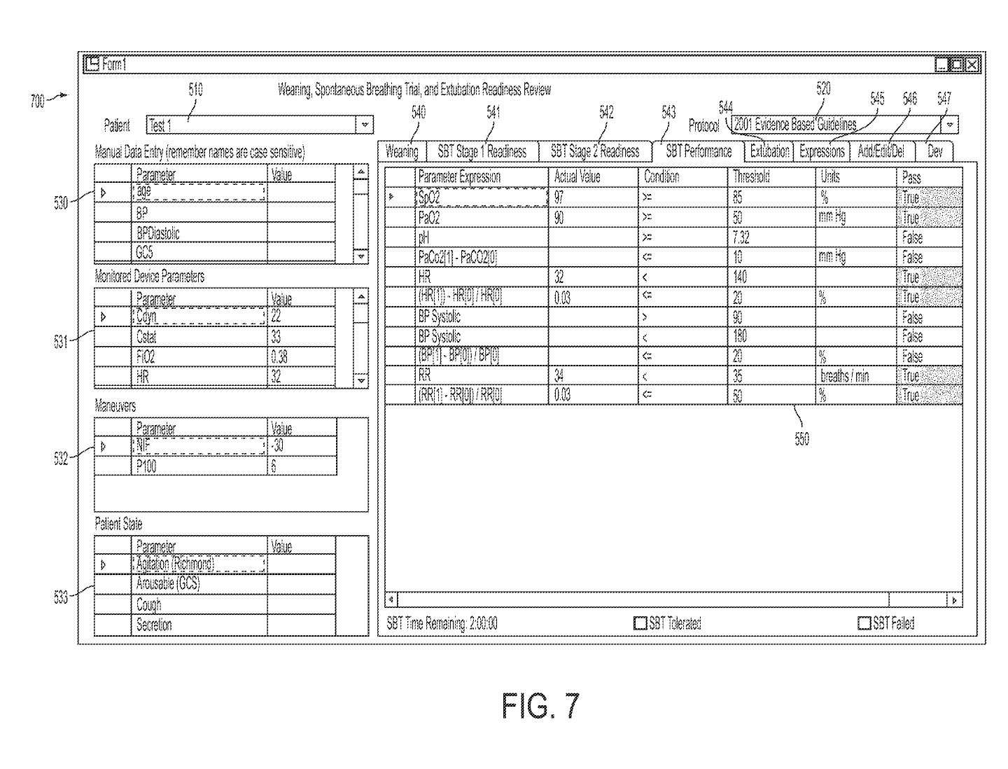

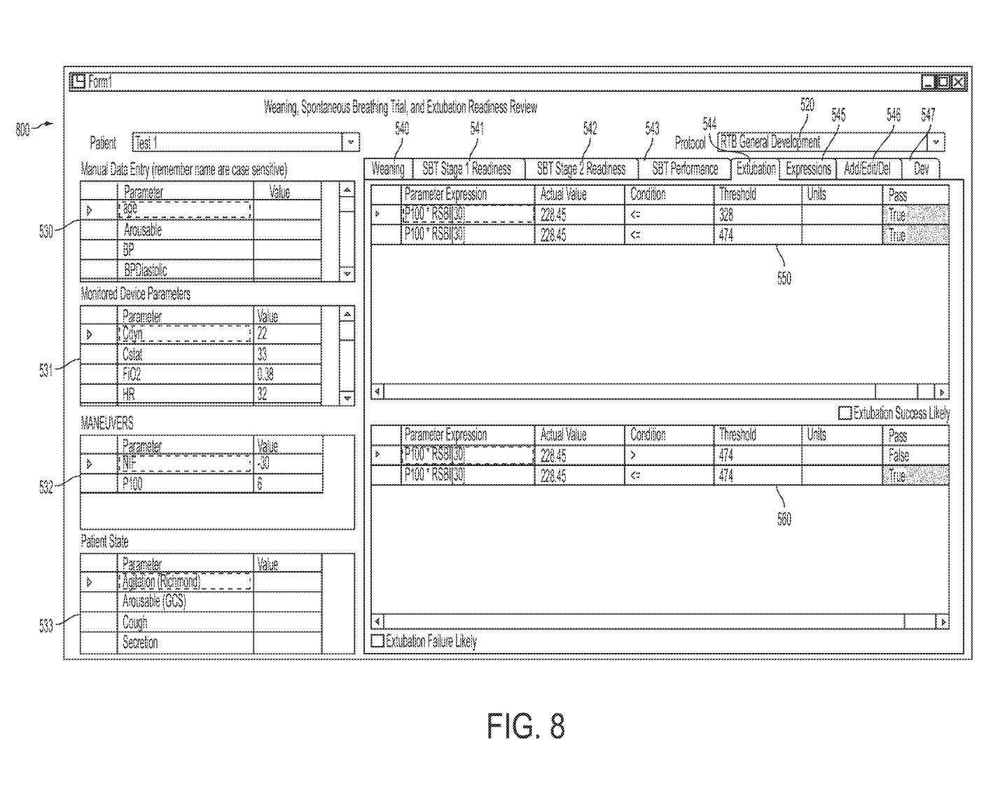

FIG. 7 presents a user interface view 700 with SBT Performance tab 543 selected. A selection tool (not shown) may be provided to enable the clinician to indicate when the SBT begins so that the system may calculate the SBT time remaining. In one example, the clinician may indicate whether the SBT was tolerated or failed by checking appropriate checkboxes. In another example, the system automatically shows a checkmark in the appropriate checkbox to indicate whether the SBT was tolerated or failed based on the success or failure of the patient parameter expressions. FIG. 8 presents a user interface view 800 with tab 544, labeled “Extubation”, selected. Extubation tab 544 includes a success likely table 550 and a failure likely table 560 as well as corresponding checkboxes. In one example, the system automatically shows a checkmark in the appropriate checkbox to indicate the likelihood of extubation success or failure based on the success or failure of the patient parameter expressions. In another example, a clinician may check the appropriate checkbox to indicate the likelihood of extubation success or failure based on the success or failure of the patient parameter expressions.

In a variation of the present embodiment, a protocol status indicator (not shown) is displayed to provide constructive notice with respect to the success or failure of the protocol. A protocol may succeed if all the expressions in the success likely table are satisfied and may fail if at least one of the expressions in the failure likely table is satisfied. In other words, success is conjunctive while failure is disjunctive. Exemplary indicators include checkboxes, color variations, images and text, and any other form of expression suitable to indicate status.

In yet another variation of the present embodiment, a patient status indicator is displayed to indicate an overall patient status.

In a still further variation of the present embodiment, a selection tool is presented (not shown) to enable a clinician to select a patient parameter or expression. The system then presents a graphical representation comprising historical values of the patient parameter or the expression.

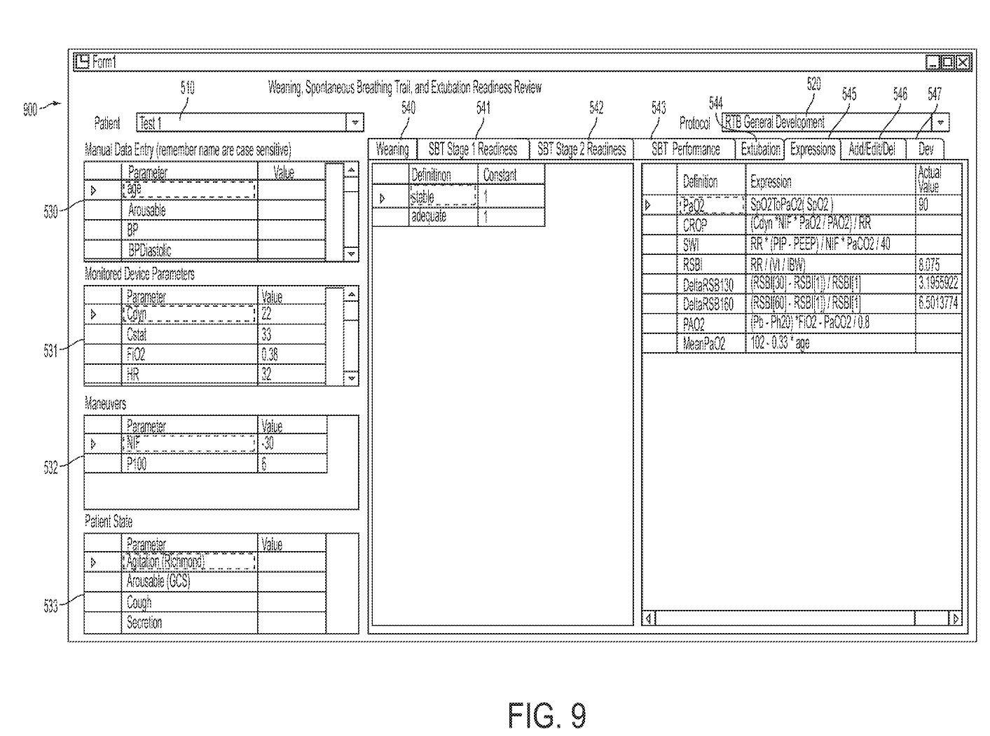

Referring now to FIG. 9, a user interface view 900 is presented with an Expressions tab 545 selected. Expressions tab 545 comprises tables of constants and variables. The constants table shows the values assigned to named constants. For example, constants named stable and adequate are assigned the value “1”. In the variables table, a plurality of variables and corresponding expressions are shown. The first variable in the table is named PaO2. The value of PaO2 is defined by the corresponding function shown next to the variable name, the PaO2 variable having an actual value equal to 90. The second variable in the table is named CROP. CROP is a composite variable in that it includes variable PaO2. The constants and variables transform parameters from data sources into forms more suitable for use in the patient parameter relationship expressions. For example, the variables may scale and normalize parameters. Also, the variables may consolidate parameters from different sources to simplify configuration of the patient parameter relationship expressions and protocols.

While this invention has been described as having an exemplary design, the present invention may be further modified within the spirit and scope of this disclosure. This application is intended to cover any variations, uses, or adaptations of the invention using its general principles. Further, this application is intended to cover such departures from the present disclosure as come within known or customary practice in the art to which this invention pertains.

What is claimed is:

1. A method to evaluate breathing stages representing levels of readiness of a patient to breathe unassisted, the method comprising:
with a user interface of a computing system, selecting one or more patient parameter relationship expression for each of a plurality of breathing stages, each of the one or more patient parameter relationship expression based on a patient parameter;
with the user interface, selecting a breathing stage from the plurality of breathing stages;
acquiring the patient parameters;
determining a status of each of the one or more patient parameter relationship expression for the selected breathing stage based on the acquired patient parameters; and
presenting a view on a display device with the user interface, the view including status indicators corresponding to the status of the one or more patient parameter relationship expression for the selected breathing stage.

2. A method as in claim 1, further comprising:
determining whether the patient is ready to advance to a succeeding breathing stage corresponding to increased readiness of the patient to breathe unassisted.

3. A method as in claim 2, wherein for at least one breathing stage, determining whether the patient is ready to advance to a succeeding breathing stage is performed by the computing system if each of the status indicators corresponding to the status of the one or more patient parameter relationship expression for the selected breathing stage indicate success.

4. A method as in claim 1, wherein the view includes a success likely table for presenting the status of at least one of the one or more patient parameter relationship expression, which is indicative of a likelihood of success of the selected breathing stage, and a failure likely table for presenting the status of at least one of the one or more patient parameter relationship expression, which is indicative of a likelihood of failure of the selected breathing stage.

5. A method as in claim 1, wherein the one or more patient parameter comprises one or more monitored parameter and one or more manual data entry parameter, and wherein acquiring the patient parameter comprises evaluating the patient and manually entering, with the user interface, the manual data entry parameter based on the evaluation.

6. A method as in claim 1, wherein the user interface is configured to present a plurality of selectable tabs corresponding to the plurality of breathing stages.

7. A method as in claim 1, wherein selecting one or more patient parameter relationship expression for each of the plurality of breathing stages comprises:
selecting, with the user interface, a protocol from a plurality of protocols, the protocol including the one or more patient parameter relationship expression.

8. A method as in claim 7, further comprising:
while in the selected breathing stage, switching the selected protocol.

9. A computing system to evaluate breathing stages representing levels of readiness of a patient to breathe unassisted, the system comprising:
a computer readable storage medium having stored therein a plurality of patient parameter relationship expressions, each of the one or more patient parameter relationship expression based on a patient parameter;
a computing device, coupled to the computer readable storage medium, that is configured to acquire at least some of the patient parameters;
a user interface, coupled to the computer readable storage medium, that is configured to (a) select one or more patient parameter relationship expression from the plurality of patient parameter relationship expressions for each of a plurality of breathing stages; (b) select a breathing stage from the plurality of breathing stages; and (c) present a view on a display device, the view including status indicators operable to indicate a status of each of the one or more patient parameter relationship expression for the selected breathing stage based on the acquired patient parameters.

10. The system of claim 9, wherein user interface is further configured to indicate whether the patient is ready to advance to a succeeding breathing stage corresponding to increased readiness of the patient to breathe unassisted.

11. The system of claim 10, wherein for at least one breathing stage, the user interface is configured to determine that the patient is ready to advance to a succeeding breathing stage if each of the status indicators corresponding to the status of the one or more patient parameter relationship expression for the selected breathing stage indicate success.

12. The system of claim 10, wherein the view includes a success likely table for presenting the status of at least one of the one or more patient parameter relationship expression, which is indicative of a likelihood of success of the selected breathing stage, and a failure likely table for presenting the status of at least one of the one or more patient parameter relationship expression, which is indicative of a likelihood of failure of the selected breathing stage.

13. The system of claim 10, wherein the one or more patient parameter comprises one or more monitored parameter and one or more manual data entry parameter, and wherein the user interface is further configured to acquire the one or more manual data entry parameter, which is based on an evaluation of the patient.

14. The system of claim 10, wherein the user interface is further configured to select a protocol from a plurality of protocols, the protocol including the one or more patient parameter relationship expression.

15. A computer program product comprising a computer readable storage medium having computer readable processing sequences embodied therein for causing a computing system to facilitate evaluation of breathing stages representing levels of readiness of a patient to breathe unassisted, the computer program product comprising:
a first processing sequence operable to select one or more patient parameter relationship expression for each of a plurality of breathing stages, each of the one or more patient parameter relationship expression based on a patient parameter;
a second processing sequence operable to select a breathing stage from the plurality of breathing stages;
a third processing sequence operable to acquire the patient parameters; and
a fourth processing sequence operable to present a view on a display device, the view including status indicators corresponding to a status of the one or more patient parameter relationship expression for the selected breathing stage.

16. A computer program product as in claim 15, further comprising a processing sequence operable to determine, for at least one breathing stage, that the patient is ready to advance to a succeeding breathing stage corresponding to increased readiness of the patient to breathe unassisted, if each of the status indicators corresponding to the status of the one or more patient parameter relationship expression for the selected breathing stage indicate success.

17. A computer program product as in claim 15, wherein the view includes a success likely table for presenting the status of at least one of the one or more patient parameter relationship expression, which is indicative of a likelihood of success of the selected breathing stage, and a failure likely table for presenting the status of at least one of the one or more patient parameter relationship expression, which is indicative of a likelihood of failure of the selected breathing stage.

18. A computer program product as in claim 15, wherein the one or more patient parameter comprises one or more monitored parameter and one or more manual data entry parameter, the computer program product further comprising a processing sequence operable to acquire the one or more manual data entry parameter, which is based on an evaluation of the patient.

19. A computer program product as in claim 15, further comprising a processing sequence operable to select a protocol from a plurality of protocols, the protocol including the one or more patient parameter relationship expression.
